(19) United States
(12) Plant Patent Application Publication
(10) Pub. No.: US 2013/0247266 P1
(43) Pub. Date: Sep. 19, 2013

(54) Helleborus plant named 'ABCRD02'

(50) ;

(71) Applicants: Rodney Davey, Devon (GB); Lynda Windsor, Devon (GB)

(72) Inventors: Rodney Davey, Devon (GB); Lynda Windsor, Devon (GB)

(73) Assignee: AMAZING BREEDERS COMPANY B.V., Aalsmeer (NL)

(21) Appl. No.: 13/385,961

(22) Filed: Mar. 16, 2012

(51) Int. Cl. A01H 5/00

(52) U.S. Cl. PLT/439

(57) ABSTRACT

A new Helleborus plant particularly distinguished by abundant purple flowers, upright plant habit and a vigorous growth habit, is disclosed.

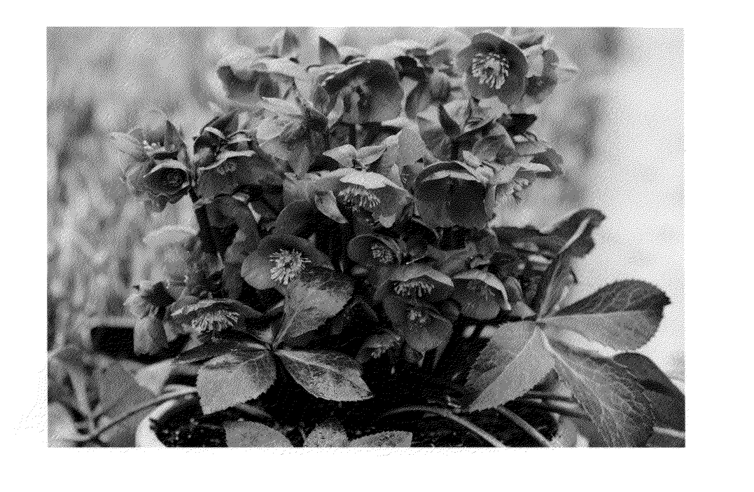

### GENUS AND SPECIES

Helleborus×hybridus

### VARIETY DENOMINATION

‘ABCRDOT’

### BACKGROUND OF THE NEW PLANT

The present invention comprises a new and distinct variety botanically known as Helleborus×hybridus, and hereinafter referred to by the variety name ‘ABCRD02’. The new variety originated from a controlled hybridization conducted in February 2005 in Devon, United Kingdom, between female hellebore plant Helleborus nr. ‘RD369HK01’ (unpatented) and male hellebore plant Helleborus nr. ‘RD959NB97’ (unpatented). The seeds produced by the hybridization were sown in Devon, United Kingdom. A single plant selection was chosen for further evaluation and for asexual propagation in Devon, United Kingdom.

‘ABCRD02’ was subsequently asexually propagated by tissue culture in Aalsmeer, The Netherlands for at least 3 years. The present invention has been found to retain its distinctive characteristics through successive asexual propagations for at least 30 generations.

Community Plant Variety Rights (CPVR) for this variety were applied for in Europe on Jan. 25, 2012. ‘ABCRD02’ has not been made publicly available more than one year prior to filing of this application.

### SUMMARY OF THE INVENTION

The following are the most outstanding and distinguishing characteristics of this new variety when grown under normal horticultural practices in Aalsmeer and Boijl, The Netherlands.

1. Abundant purple flowers;

2. Upright plant habit; and

3. A vigorous growth habit.

### DESCRIPTION OF THE PHOTOGRAPH

This Helleborus plant is illustrated by the accompanying photograph which shows overall plant habit including blooms, buds, and foliage of the plant; the colors shown are as true as can be reasonably obtained by conventional photographic procedures. The photograph is of a whole plant about one-year-old, grown indoors in a glass covered greenhouse in Boijl, The Netherlands and Aalsmeer, The Netherlands.

### DESCRIPTION OF THE NEW VARIETY

The following detailed description sets forth the distinctive characteristics of ‘ABCRD02’. The data which define these characteristics were collected from asexual reproductions carried out in Aalsmeer and Boijl, The Netherlands. The plant history was taken on one-year-old plants grown indoors. Color readings were taken under artificial light. Color references are primarily to the R.H.S. Color Chart of The Royal Horticultural Society of London (R.H.S.) (2005).

### DETAILED BOTANICAL DESCRIPTION

Classification:
   Family.—Ranunculaceae.
   Genus and species.—Helleborus×hybridus.
   Common name.—Christmas Rose.
   Variety name.—‘ABCRD02’.
Plant:
   Plant and growth habit.—Upright and vigorous.
   Propagation.—In vitro propagation by tissue culture.
   Height.—25.0 cm to 35.0 cm.
   Width.—25.0 cm.
   Time to initiate roots.—6 weeks.
   Time to produce a rooted cutting.—Approximately 3 months.
   Time to produce a finished plant.—3 months.
Stems:
   Shape.—Compact.
   Length.—3.0 cm.
   Diameter.—2.0 cm.
   Internode length.—0.1 cm.
   Texture.—Smooth.
   Color.—RHS 139A.
Leaves:
   Type.—Palmately compound.
   Arrangement.—Alternate.
   Length.—20.0 cm to 25.0 cm.
   Width.—15.0 cm to 20.0 cm.
   Shape.—Ovate to lanceolate.
   Apex.—Obtuse.
   Base.—Lanceolate.
   Margin.—Serrate.
   Texture (both upper and lower surfaces).—Smooth.
   Color.—Immature leaf: Upper surface: RHS 139A Lower surface: RHS 147A Mature leaf: Upper surface: RHS 139A Lower surface: RHS 147A.
   Venation pattern.—Longitudinal.
   Venation color (both upper and lower surfaces).—RHS 137C.
   Variegation.—Absent.
   Petiole.—Length: 20.0 cm Diameter: 0.6 cm Color: RHS 147A and RHS 184B Texture: Smooth.
   Petiolule.—Length: 1.5 cm Diameter: 0.5 cm Color: RHS 147A and RHS 183C Texture: Smooth.
   Stipules.—Absent.
Inflorescence:
   Type.—Loosely branched cymes.
   Shape.—Loosely branched.
   Self cleaning or persistent.—Persistent.
   Flowering habit.—Vertical facing flowers above the foliage.
   Flowering season.—January through March.
   Quantity of inflorescence on the plant.—8 to 10.
   Fragrance.—Absent.
   Lastingness of inflorescence on the plant.—At least 2 months.
   Diameter.—15.0 cm.
   Depth (height).—25.0 cm to 30.0 cm.
Bud:
   Height.—2.5 cm.
   Diameter.—1.5 cm.
   Shape.—Ovate.
   Color.—RHS N79C.
   Texture.—Smooth.
Bracts:
   Shape.—Ovate.
   Quantity per inflorescence.—2 to 10.
   Length.—3.0 cm to 8.0 cm.
   Width.—2.0 cm.
   Apex.—Acute.
   Base.—Ovate.
   Margin.—Smooth.
   Texture (both upper and lower surfaces).—Smooth.
   Color.—Upper surface: RHS 139A Lower surface: RHS 147A.
Peduncle:
   Shape.—Round.
   Length.—15.0 cm.
   Width.—0.5 cm.
   Texture.—Smooth.
   Color.—RHS 187B.
Pedicel:
   Length.—4.0 cm to 10.0 cm.
   Diameter.—0.3 cm.
   Texture.—Smooth.
   Color.—RHS 187B.
Sepals:
   Quantity per flower.—5.
   Shape.—Orbicular.
   Length.—4.0 cm.
   Width.—3.0 cm.
   Apex.—Rounded.
   Base.—Rounded.
   Margin.—Smooth.
   Texture (both upper and lower surfaces).—Smooth.
   Color (both upper and lower surfaces).—RHS N79C.
Reproductive organs:
   Stamens.—Quantity per flower: Approximately 60 Color: RHS 155D Filament length: 1.0 cm Filament width: 0.05 cm.
   Anthers.—Shape: Ovate Length: 0.2 cm Diameter: 0.1 cm Color: RHS 157B Pollen amount: Sparse Pollen color: RHS 157B.
   Pistils.—Quantity per flower: 5 Shape: Tuberculate Length: 2.5 cm Width: 0.1 cm Color: RHS N79C.
   Stigma.—Length: 0.2 cm Width: 0.05 cm Color: RHS N79C.
   Ovary.—Shape: Ovate Color: RHS 139A.
Fruit and seed set: None observed
Disease and insect resistance: No particular resistance or susceptibility observed
Physiological characteristics: Frost tolerant to 15° C.

### COMPARISON WITH COMMERCIAL VARIETY

‘ABCRD02’ differs from Helleborus variety ‘ABCRD01’ (U.S. Plant patent application No. 13/374,191) in that ‘ABCRD02’ has a flower bud color of RHS N79C (purple), whereas ‘ABCRD01’ has a lighter flower bud color of RHS 70B (red-purple). Additionally, ‘ABCRD02’ has both an upper and lower surface sepal color of RHS N79C (purple), whereas ‘ABCRD01’ has an upper and lower surface sepal color of RHS 70B (red-purple) and RHS 70C (red-purple), respectively.

We claim:

1. A new and distinct variety of Helleborus plant as described and shown herein.
